(19) United States
(12) Patent Application Publication
(10) Pub. No.: US 2025/0295860 A1
(43) Pub. Date: Sep. 25, 2025

(54) SENSOR ASSEMBLY FOR AN INJECTION DEVICE

(71) Applicant: Sanofi, Paris (FR)

(72) Inventor: Michael Helmer, Frankfurt am Main (DE)

(21) Appl. No.: 18/862,422

(22) PCT Filed: May 2, 2023
(86) PCT No.: PCT/EP2023/061438

§ 371 Date: Nov. 1, 2024

(30) Foreign Application Priority Data
May 3, 2022 (EP) 22315094.7

(51) Int. Cl. A61M 5/315

(52) U.S. Cl. CPC A61M 5/31566; A61M 5/31546; A61M 2205/3317; A61M 2205/505

(57) ABSTRACT

The disclosure relates to a sensor assembly for an injection device, the sensor assembly including-a sensor element attachable to the injection device and including a sensing surface, the sensing surface including a touch sensitive sensor segment, the touch sensitive sensor segment being operable to generate or to modify an electric touch signal when touched by a body part of a user, a processor connected to the sensor element, operable to detect a variation of the electric touch signal over time and to generate a control signal on the basis of the temporal variation of the electric touch signal, and a transmitter connected to the processor and operable to transmit the control signal to an external electronic device.

### CROSS-REFERENCE TO RELATED APPLICATIONS

The present application is the national stage entry of International Patent Application No. PCT/EP2023/061438, filed on May 2, 2023, and claims priority to European Application No. EP 22315094.7, filed on May 3, 2022, the disclosures of which are incorporated herein by reference.

### TECHNICAL FIELD

The present disclosure relates to a sensor assembly for an injection device and to an injection device equipped with such a sensor assembly. In a further aspect the disclosure relates to an add-on device configured for fastening to an injection device, wherein the add-on device is provided with a sensor assembly.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is beneficial to assist the user by making use of an external electronic device, such as a mobile electronic device, e.g. implemented as a smartphone, a tablet computer, or a smart watch. A software application provided on such external electronic devices may interact with the user and may provide instructions or recommendations to the user of how to correctly use the injection device.

User assistance provided by an external electronic device may require input from the user, which in the course of administering a medicament, e.g. by way of injection, might be somewhat complicated or cumbersome for the user. There might be scenarios of use, where a user holds the external electronic device in one hand while keeping the injection device in the other hand. In such a situation, entering a command or a confirmation on the external electronic device while holding the injection device in that particular hand used that can be used for entering such a command may also come along with a non-neglectable risk of an unintended stitch damage.

It is therefore desirable to provide an easy and intuitive way of how to assist a user in using an injection device when making additional use of an external electronic device. It is a further aim to provide an intuitive and rather easy monitoring or logging of an operation of an injection device.

### SUMMARY

In one aspect, the present disclosure relates to a sensor assembly for an injection device. The sensor assembly includes a sensor element attachable to the injection device and including a sensing surface. The sensing surface includes a touch sensitive sensor segment. The touch sensitive sensor segment is operable to generate or to modify an electric touch signal when touched by a body part of a user. The sensor element typically includes a one-dimensional or two-dimensional sensing surface.

The sensor assembly further includes a processor connectable or connected to the sensor element. The processor is operable to detect a variation of the electric touch signal over time and to generate a control signal on the basis of the temporal variation of the electric touch signal.

The sensor assembly further includes a transmitter connected to the processor and operable to transmit the control signal to an external electronic device.

This way, the sensor assembly may be operable to detect a contact with a body part of a user, to generate a control signal, which is indicative of the contact with the user and to transmit the control signal to an external electronic device. Accordingly, the sensor assembly, which is attachable to the injection device or which might be integrated into the injection device may provide a kind of a remote control for the external electronic device.

A user holding the injection device, e.g. in one hand, may easily reach the touch sensitive sensor element to generate the control signal and to transmit the control signal to the external electronic device. In some examples the sensor assembly is attached to a proximal end of the elongated injection device, such that it is easily reachable e.g. by a thumb of the user while holding the injection device with the palm and/or in further fingers of the same hand.

Typically, generation of the control signal by the processor is triggered by the detection of a variation of the electric touch signals over time. Submission of the control signal to the external electronic device may be automatically triggered upon generating the control signal.

In effect, by touching the sensor element of the sensor assembly the control signal will be generated and automatically transmitted to the external electronic device.

According to some embodiments of the present disclosure, the sensing surface of the sensor assembly includes a number of touch sensitive sensor segments. Each of the sensor segments is operable to generate or to modify an electric touch signal when touched by the body part of the user. The sensor segments are spatially separated on the sensing surface in a non-overlapping way. They may be arranged next to each other in a regular or irregular manner. In some examples the entire sensing surface is covered and/or occupied by numerous sensor segments. The sensor segments may be of equal or of unequal size.

In some examples, the touch sensitive sensor segments are decoupled from each other. Each touch sensitive sensor segment may operate independent from any other touch sensitive sensor segment of the sensing surface. Hence, then touch sensitive sensor segments may be operable to generate electric touch signals individually when touched by the body part of the user.

The sensor segments may belong to a touch sensitive matrix or may constitute a touch sensitive matrix, e.g. a one or two-dimensional array of touch sensitive segments. The touch sensitive segments may each include a capacitor or resistor, that is operable to generate or to modify an electrical signal in response to a physical contact, e.g. with a body part of a user. Insofar, the plurality of touch sensitive sensor segments forms or constitutes a spatially resolving touch sensitive sensor.

In further examples the touch sensitive surface or touch sensitive matrix includes a matrix of electrically resistive elements, which change their electrical and measurable resistance when e.g. touched by a user. With other examples the touch sensitive surface or touch sensitive matrix includes a matrix of capacitive elements operable to change their measurable electric capacitance when e.g. touched by a user.

In yet further examples the touch sensitive surface or touch sensitive matrix includes a combination of electrically resistive sensor segments and capacitive sensor segments. Capacitive sensors are exhibit a rather low degree of electric power consumption.

In still further examples the touch sensitive surface or touch sensitive matrix is based on surface acoustic wave technology that relies upon soundwaves. Accordingly, the touch sensitive surface or touch sensitive matrix includes at least one pair of an acoustic wave transducer and an acoustic wave receiver.

In another example the touch sensitive surface or touch sensitive matrix includes a number of optical sensors, such as photodetectors or photodiodes.

In yet another example, the touch sensitive surface or touch sensitive matrix includes ultrasonic sensors. Optical sensors and/or ultrasonic sensors could be also implemented as fingerprint sensors being capable to distinguish a characteristic fingerprint of a first user from a characteristic fingerprint of a second user.

In some examples the sensor element is attachable to a user actuatable portion of the injection device. It may be attachable or may be attached to a surface of an actuation element or handling element, which actuation element or handling element is usually touched, handled or actuated by a user when using or operating the injection device. The sensor element may be also integrated into a surface of the actuation element or handling element. The user actuatable portion may be a portion of at least one of a sleeve-shaped housing component, a trigger or a dose dial of the injection device.

By providing a number of sensor segments on or across the sensing surface a spatially resolved contact sensing can be provided. Hence, the processor connected to the respective touch sensitive sensor segments is operable to identify those sensor segments that generate or modify an electric touch signal in response to a mechanical contact to the body part of the user. This way, the processor is operable to determine which section or subsection of the sensing surface is actually subject to a mechanical contact with the body part of the user. This allows to provide a spatially resolved touch sensing of the body part on the sensing surface of the sensor element.

According to a further example the processor of the sensor assembly is operable to generate the control signal on the basis of the temporal variation of a plurality of electric touch signals generated or modified by numerous touch sensitive sensor segments. This way, the sensor is operable to detect and/or to provide a spatial profile as well as a spatial-temporal profile of individual touch sensitive sensor segments being touched by the body part of the user. Hence, the processor may be operable to ascertain and/or to detect a region on the sensing surface that is touched by the body part. The processor may be further operable to monitor a temporal modification or movement of such a region over time. Here, a region touched by the body part may be also regarded and/or denoted as a sensing area of the sensing surface.

According to a further example the touch sensitive sensor segment and/or some or each one of the plurality of touch sensitive sensor segments is operable to generate different electric touch signals in response to a variation of a pressure applied to the touch sensitive sensor segment. Insofar, the electric touch signals to be generated by a touch sensitive sensor segment vary with the pressure e.g. applied by a body part of a user getting in touch or getting in contact with this particular touch sensitive sensor segment.

In response to a variation of the pressure applied to the touch sensitive sensor segment the electric touch signal may differ in magnitude or amplitude. Furthermore, the electric touch signal may change its sign or may change its frequency or periodicity. In response to a varying pressure applied to the touch sensitive sensor segment the respective sensor segment is operable to vary the electric touch signal in a measurable way, i.e. in a way that is detectable or processable by the processor connected or connectable to the touch sensitive sensor segment.

In some examples, at least some or all touch sensitive sensor segments of the sensing surface are operable to generate different electric touch signals in response to a variation of the pressure applied thereto.

For example, the touch sensitive sensor segment may be operable to generate at least two different electric touch signals in response to a variation of a pressure applied thereto. By default the touch sensitive sensor segment may be operable to generate a first electric touch signal in response to a first pressure applied to the touch sensitive sensor segment. Here, a first electric touch signal may be generated if the first pressure applied to the touch sensitive sensor segment is above a first predefined threshold.

The touch sensitive sensor segment may be further operable to generate a second electric touch signal that differs in at least one of a magnitude, an amplitude, a sign or a frequency from the first electric touch signal. The second electric touch signal may be generated when the pressure applied to the touch sensitive sensor segment is at or above a second predefined threshold. Typically, the second threshold is above the first threshold.

Insofar, the first and second electric touch signals may be indicative of a rather low and a rather high pressure applied to the respective touch sensitive sensor segments, respectively.

In some examples, the touch sensitive sensor segment or a plurality of sensor segments is or are operable to generate a variety of different electric touch signals. It is conceivable that the touch sensitive sensor segment is operable to generate at least three, at least four, at least five, at least six or at least eight different electric touch signals, each of which reflecting or indicating a respective first, second, third, fourth, fifth, sixth or eights pressure applied to the respective touch sensitive sensor segment.

The touch sensitive sensor segment may be operable to generate an electric touch signal that varies gradually with a variation of the pressure applied to the respective touch sensitive sensor segment. Here, the electric touch signal may be directly representative of the applied pressure.

Depending on the number and size of touch sensitive sensor segments distributed across the sensing surface there can be provided a precise and rather detailed spatially resolved pressure profile of or across the sensing surface of the sensor element. In this way, the sensor assembly is operable to detect or to measure a spatially resolved pressure profile applied across the sensing surface by a body part of a user.

The spatially resolved pressure profile and/or a temporal variation of such a spatially resolved pressure profile may be indicative of a particular touch procedure or gesture conducted by the user of the injection device.

In some examples, the processor is further operable to generate a variety of different control signals depending on the respective electric touch signal received from the touch sensitive sensor segment. Here, the processor is operable to generate and/or to transmit a first control signal when receiving a first electric touch signal. The processor may be further operable to generate and/or to transmit a second control signal when receiving a second electric touch signal. First and second control signals differ from each other and may result in different actions or configurations of the external electronic device when received by the external electronic device.

This way, and in some examples the control signal transmitted to the external electronic device may be indicative of a pressure level applied to a single or to a plurality of touch sensitive sensor segments. In further examples the control signal transmitted to the external electronic device may be indicative of a size of a sensing area in contact with the body part of the user. In further examples the control signal transmitted to the external electronic device may be indicative of a position, a movement and/or temporal variation of a sensing area in contact with the body part of the user. In further examples, the control signal may be indicative of a combination of the above-mentioned parameters, namely the pressure level, the size of the sensing area, and/or the position and/or movement of the sensing area on the sensing surface.

In this way and with a single sensing surface there can be generated and transmitted a plurality of different control signals to be processed by the external electronic device.

According to a further example the processor is operable to assign the temporal variation of the electric touch signal to one of a plurality of predefined user gestures. The processor is further operable to generate the control signal by selecting the control signal from a plurality of predefined control signals on the basis of the user gesture assigned to the temporal variation of the electric touch signal. In some examples the processor may be operable to detect different user gestures and/or to distinguish between different user gestures.

In some examples the processor may be operable to distinguish between numerous user gestures, such as a swiping across the sensing surface, a single tapping of the sensing surface and a twofold or multiple tapping of the sensing surface. The processor may be further operable to distinguish between a short-term tapping of the sensing surface, e.g. a tapping that lasts less than 1 second and a long-term tapping of the sensing surface, e.g. a tapping that lasts more than 1 or 2 seconds.

Furthermore, the processor may be operable to distinguish between a slight tapping and a firm or hard tapping of the sensing surface. This way, the sensor assembly is configured to detect or to recognize a gesture of the body part of the user. Recognizing or detecting different user gestures by way of the touch sensitive sensing surface of the sensor assembly allows to implement a rather space saving and intuitive on-board remote control for an injection device, which remote control is configured to control and/or to interact with an external electronic device.

According to a further example, the processor is operable to recognize at least one of a swipe motion of the body part across the sensing surface, a short-term tapping motion of the body part on the sensing surface, a multiple short-term tapping motion of the body part on the sensing surface, a long-term tapping motion of the body part on the sensing surface and a variable pressure applied by the body part on the sensing surface. These motions may represent a single gesture, or multiple gestures conducted by the body part of the user on or with the sensing surface.

In further examples the processor may distinguish between different gestures conducted by the body part on the sensing surface. Each of the above-mentioned gestures may be defined by a spatial and/or temporal profile of electric touch signals generated by one or several touch sensitive sensor segments. The above-mentioned gestures may be defined by a reference profile of electric touch signals, e.g. stored in a memory of the sensor assembly.

In a recognizing or sensing mode of the sensor assembly the electric touch signals obtained from the touch sensitive sensor segment(s) may be compared with the stored reference profiles. Here, the processor may conduct a best matching comparison in order to assign one of a stored reference profile to a profile generated or derived on the basis of the electric touch signals actually measured or detected. The reference profile assigned to the profile of electric touch signals actually measured may be then indicative of a gesture conducted by the body part of the user on the sensing surface. The control signal generated by the processor and transmitted to the external electronic device may be indicative of the gesture determined or detected by the processor of the sensor assembly.

According to a further example, the processor is operable to process electric touch signals of a number of touch sensitive sensor segments to ascertain a sensing area of the sensing surface being touched by the body part. Typically, and when using a finger as a body part to get in contact with the sensing surface of the sensor element the respective touch sensitive sensor segments will be able to detect a pressure applied by the respective body part. In this way all touch sensitive sensor segments getting in mechanical contact with the body part, e.g. during or for operation of the injection device may generate a respective electric touch signal.

By simultaneously processing the signals of the touch sensitive sensor segments the processor may provide or may ascertain a sensing area on the sensing surface that is actually in touch or in mechanical contact with the body part of the user. In this way, the processor is operable to detect, whether a middle part or a border region of the sensing surface is actually touched by the body part.

Moreover, the processor may be operable to detect or to measure a size of the sensing area. By ascertaining or determining a sensing area of the sensing surface being actually touched or being in mechanical contact with the body part of the user the sensor assembly provides a rather precise and spatially resolved monitoring of how the body part of the user touches the sensor element.

According to a further example, the processor is operable to detect a movement of the sensing area on the sensing surface. Here, the processor may e.g. detect a swiping motion of a finger across the sensing surface. In further situations, the body part of the user itself may be subject to a movement during an operation of the injection device, e.g. during setting of a dose or during injection of a dose the movement of the body part e.g. relative to a housing of the injection device may be accompanied by a measurable movement of the body part relative to the sensor element and hence relative to the sensing surface. Here, the body part of the user, e.g. a finger or a thumb may be subject to a milling motion or a natural rolling motion during the operation of the injection device.

Such a movement of the body part relative to the sensor element may lead to a movement of the sensing area on the sensing surface that is touched by the body part. A movement of the body part relative to the sensor element may induce an activation and deactivation of numerous touch sensitive sensor segments spatially distributed across the sensing surface of the sensor element. This leads to a measurable change of a variation of electric touch signals and to a respective variation of the sensing area measurable by the processor.

By evaluating and processing the electric touch signals generated by the touch sensitive sensor segments during a respective operation of the injection device the processor recognize or characterize a particular operation of the injection device. Here, the sensor assembly and hence its processor may be operable to distinguish between different operation modes of the injection device by evaluating a measurable movement of the sensing area on the sensing surface.

According to a further example, the processor of the sensor assembly is operable to detect a variation of a size of the sensing area on the sensing surface. A variation of a size of the sensing area may result from a variation of the pressure applied by the body part onto the sensor element. Since the body part may include a certain elasticity and may further include a rather convex-shaped outside facing structure or surface, by increasing a pressure applied by the body part onto the sensor element the fraction of the body part getting in direct contact with the sensing surface may increase, e.g. due to an elastic deformation of the body part. This may lead to an increase of the size of the sensing area on the sensing surface.

A detection of a variation of the size of the sensing area during an operation of the injection device may be hence indicative for a variation of the pressure applied by the user onto the sensor element. In some examples, the size variation measurable by the processor as well as a variation of the type, magnitude, amplitude, sign or frequency of the electric touch signals generated by the touch sensitive sensor segments may be processed in a combined manner. Insofar, a variation of the pressure applied by the body part to the sensor element may be detected by a variation of the size of the sensing area concurrently with a variation of the electric touch signals generated by the pressure sensitive and touch sensitive sensor segments.

Here, a variation of the pressure applied by the body part may be monitored or detected in a twofold manner. Hence, a variation of a pressure applied to the sensor element can be measured or determined in at least two different ways thereby increasing or providing a redundancy of the measurement system provided by the sensor assembly.

According to a further example, the processor is operable to detect a variation of a geometry and/or to detect a variation of an orientation of the sensing area on the sensing surface. In this way, further modes of operation, during which the body part of the user is subject to a movement or change relative to the sensor element can be detected and evaluated. Variations of a geometry or orientation of the sensing area on the sensing surface, which is typically detected by a respective variation of electric touch signals generated by a number of touch sensitive sensor segments may be further indicative of particular scenarios of use of the injection device.

By electronically detecting and/or by electronically and quantitatively measuring at least one of a movement of the sensing area, a variation of a size of the sensing area, a variation of a geometry and/or a variation of an orientation of the sensing area on the sensing surface the sensor assembly is operable to detect, to recognize, to characterize and/or to measure different operations and operation modes of the injection device. It may be further operable to detect, recognize, characterized and/or measure different control operations or gestures conducted by a body part of the user on or with respect to the sensing surface.

According to a further example, the sensor element includes a planar sensing surface configured for fastening to an end face of a trigger of the injection device. The planar sensing surface may include a number of touch sensitive sensor segments located adjacently next to each other on the planar sensing surface. In some examples the entire planar sensing surface may be filled or occupied with adjacently arranged sensor segments. Insofar, the entire planar sensing surface may be implemented as a touch sensitive sensing surface of the sensor element.

The planar sensing surface may cover the entirety of an end face of a trigger of the injection device. Typically, the trigger is configured to be depressed by a finger of a user to initiate and/or to control an injection procedure conducted by the injection device. By applying or attaching the planar sensing surface on the depressible end face of the trigger the sensor element and hence the entire sensor assembly may be integrated into the trigger of the injection device.

By arranging the sensing surface on an end face of the trigger of the injection device, the sensing surface is easily accessible to e.g. a thumb of a user's hand that holds the injection device while the external electronic device may be held in another hand of the user. When the sensor assembly is implemented as a kind of a remote control for the external electronic device the user may easily provide respective control commands to the external electronic device by transmitting the control signal to the external electronic device via the transmitter.

In another example, the planar sensing surface may be configured for fastening to an end face of an auxiliary trigger of an add-on device configured for fastening to an injection device. The auxiliary trigger may mimic the trigger of the injection device and may operably engage with the trigger of the injection device when the add-on device is attached or fastened to the injection device. Here, the auxiliary trigger may replace or substitute the functionality of the original trigger of the injection device.

The add-on device may cover the trigger section of the injection device. The auxiliary trigger may be in direct or indirect operable engagement with the trigger of the injection device, such that by depressing or actuating the auxiliary trigger of the add-on device the trigger of the injection device will be actuated or depressed, respectively. With an implementation of the sensor assembly to or into an add-on device the planar sensing surface may be fastened or integrated to an end face of such an auxiliary trigger of the add-on device.

In some examples the planar sensing surface may be integrated into the end face of the trigger or auxiliary trigger. It may be fastened to the end face of the trigger or may be integrated into the end face of the trigger. By attaching or integrating the planar sensing surface to the end face of the trigger there can be provided a rather integrated solution for implementing or integrating the sensor assembly into or onto the injection device or at an add-on device.

According to a further example, the sensor element includes a tubular shaped sensing surface configured for fastening to a tubular member of the injection device. With this example the sensor element may be configured to enclose or to at least partially enclose a tubular shaped structure or tubular member of the injection device, such as a dose dial or a housing component of the injection device.

In some examples, the tubular shaped sensing surface of the sensor element is configured to enclose an outside surface of the tubular shaped dose dial of the injection device. A dose of individual size of the injection device may be set by the user using the dose dial and rotating the dose dial either in a dose incrementing direction or dose decrementing direction relative to the body or housing of the injection device. By having the tubular shaped sensing surface mounted or integrated on the outside of the dose dial such a dose dialing procedure can be precisely detected, recognized, characterized or even quantitatively measured by the sensor assembly.

In other examples, the tubular shaped sensing surface is attachable or fixable to an outside surface of a tubular shaped housing component of the injection device. The housing component may be clasped by a hand, e.g. by a palm or by numerous fingers of a user of the injection device for preparing and/or for conducting a dose injection procedure. Also here, and by having the sensor element on the outside surface of the tubular shaped body or housing of the injection device the user induced handling or operation of the injection device can be precisely detected, characterized, recognized or measured.

Also, the sensor element provided on the outside surface of the tubular-shaped body or housing of the injection device is easily accessible by e.g. a finger or thumb of a user's hand that holds the injection device. By touching the sensing surface and/or by swiping over the sensing surface the user may generate and trigger transmission of a control signal to the external electronic device.

According to a further example, the sensor element includes a planar sensing surface and further includes a tubular shaped sensing surface. The sensor element may include a cup-shaped structure including the planar sensing surface at a longitudinal end adjoining a tubular-shaped sensing surface forming a sidewall of the cup-shaped sensor element. Such a sensor element may be operable or configured for attaching to a dose member of an injection device, wherein the dose member is implemented as a combined dose dial and trigger. Here, by rotating the dose member a dose of variable size may be set and by depressing the dose member the injection process may be triggered and/or controlled.

According to a further example, the sensor element includes a flexible sheet configured for wrapping around the tubular member of the injection device. The sensor element may include a planar substrate made of a flexible sheet, e.g. made of a flexible foil. The planar substrate may be pliable and/or foldable or wrappable into a tubular shape.

The flexible sheet or substrate may be provided with numerous touch sensitive sensor segments. The pliable or flexible sheet enables to wrap around the tubular member of the injection device. A flexible sheet of the sensor element and the touch sensitive sensor segments attached or mounted on the flexible sheet allows to use the sensor element with a large variety of differently sized tubular members of the injection device.

The sensor element may be universally applicable to tubular members of different size. By way of the flexible sheet the entire sensor element may be flexible and may be easily fixable to the tubular member of the injection device. It may be adhesively attached to an outside surface of the tubular member and may provide a spatially resolving touch sensitive area on the outside of the tubular member, e.g. on the outside of the housing of the injection device.

The same may also apply to an add-on device presumed that the add-on device includes a tubular member, e.g. for fastening or clasping around the housing of the injection device.

According to a further example, the tubular member of the injection device is a dose dial rotatable relative to a body of the injection device for setting of a dose. By attaching the flexible sensor element to the dose dial a dose dialing or dose setting operation conducted by a user of the injection device, thereby rotating the dose dial relative to the body or housing of the injection device can be detected, recognized, characterized or quantitatively measured by the sensor assembly.

According to a further example, the tubular member is a body of the injection device. In some examples the body of the injection device includes an elongated tubular sleeve. The body may be sized to accommodate a drive mechanism for expelling or withdrawing a dose of a medicament from a medicament container. In some examples the medicament or medicament container, e.g. implemented as a syringe or cartridge containing the liquid medicament, may be also arranged inside the body or housing of the injection device.

According to a further example, the tubular member is a clip configured for detachably fastening to the injection device. Here, the tubular member may be implemented as a part of an add-on device configured for fastening to the housing of the injection device.

According to a further example, the processor of the sensor assembly is operable to detect, to recognize, to characterize and/or to measure an operation of the injection device on the basis of the temporal variation of the electric touch signal. The processor is further operable to generate the control signal by selecting one of a number of predefined control signals being indicative of the detected, recognize, characterized and/or measured operation of the injection device. With this example it is of particular benefit when the sensor assembly and hence the sensor element is attached to an operating element of the injection device, such as a dose dial, a trigger and/or a housing of the injection device. The operating element is typically actuated or operated by a user to conduct or to trigger a particular device operation, such as setting of a dose, dispensing or injecting of a dose and/or keeping the injection device in an end of dose configuration, in which the injection needle after termination of the injection procedure remains in the skin for a predefined time interval.

Insofar, the processor may be operable to detect at least one of a dose setting operation of the injection device, a dose dispensing operation of the injection device and a holding operation of the injection device by processing numerous electric touch signals of numerous touch sensitive sensor segments over time when the sensor element is attached to the injection device or when the sensor element is indirectly attached to the injection device, e.g. by an auxiliary device or add-on device configured for fastening to the injection device.

A dispensing operation, e.g. the start, the duration and/or the end of a dispensing operation can be typically detected, recognized or characterized by processing of electric touch signal generated or modified by touch sensitive sensor segments of the sensing surface, which may be provided on an end face of the trigger of the injection device or on the respective end face of an auxiliary trigger of an add-on device.

Moreover, an end of dose holding operation of the injection device, i.e. a period of time, during which the user should apply and maintain a certain pressure on the trigger of the injection device after termination of the dose injection procedure, can be precisely monitored. Here, and during the holding operation at the end of a dose injection procedure the electric touch signals generated or modified by the numerous touch sensitive sensor segments should be rather constant.

According to a further example, the processor is operable to distinguish between a dose setting operation, a dose dispensing operation and an end of dose holding operation of the injection device by processing numerous electric touch signals of numerous touch sensitive sensor segments over time when the sensor element is attached to the injection device or to the add-on device, which add-on device itself is then attached to the injection device. Each operation mode of the injection device can be characterized by a temporal and/or spatial profile of numerous touch sensitive sensor segments being touched by the body part of the user during the respective operation mode.

By evaluating such a temporal and/or spatial profile measurable by the sensor assembly the sensor assembly is and becomes operable to automatically distinguish between different operation modes of the injection device. Insofar, the sensor assembly can be provided with an automated operation mode detection of the injection device thus simplifying the use of the sensor assembly and increasing the precision of a continuous injection monitoring or injection logging over time.

According to a further example, the sensor assembly includes a clock or a clock generator connected to the processor. This way, the processor is operable to detect or to measure at least one of a point of time, at which a user operates the injection device, and a duration during which a user operates the injection device. By providing the processor with a clock, respective points of time or time durations can be detected or logged, at which the processor detects a particular electric touch signal or a variation of a sensing area being indicative e.g. of a start or termination of at least one of a dose setting operation and a dose dispensing operation.

According to a further example, the sensor assembly includes a memory connected or coupled to the processor. The processor is operable to store use related data in the memory, wherein the data contains at least one of a point of time at which the injection device was operated, the duration during which the device was operated and a size of a dose of the medicament set or injected by the injection device. All these parameters, points of time, time durations and dose size information can be derived from a variation of the electric touch signals or on the basis of a variation of the sensing area detectable by the processor connected to the sensor element.

According to a further example, the processor, the clock and the memory may be operable to automatically store or to automatically monitor and/or to log a sequence of user-induced operations of the injection device only by detecting or measuring variations of electric touch signals or variations of the sensing area of the sensor element.

According to a further example, the processor may be provided with a wake-up function. Here, the processor may automatically switch into a sleep mode, e.g. when determining that the sensor element and its sensing surface has not been touched over a predefined time interval. Upon detection of an initial contact with the sensing surface of the sensor element the processor may switch into an activated mode and may wake up accordingly. By providing the processor with a sleep functionality energy provided to the sensor assembly can be saved. Battery lifetime of the sensor assembly can be hence prolonged accordingly.

According to a further example, the sensor assembly includes a power source, e.g. in form of an electric power source, such as a battery.

In further examples, the transmitter of the sensor assembly includes a transceiver. The transceiver may be operable to communicate with the external electronic device in a bi-directional way. The transceiver may provide transmission of the control signal to the external electronic device as well as receiving signals from the external electronic device. This way, the sensor assembly may receive e.g. a request signal from the external electronic device, which request signal is provided to and processed by the processor. In response to receive a request signal the processor may generate a specific control signal and transmit the control signal to the external electronic device as a response to the request signal.

The transmitter and/or transceiver may be implemented as a wireless transceiver. The transceiver may be operable to establish a communication link with the external electronic device. In some examples the transceiver may be operable to harvest energy from the external electronic device. Insofar, the transceiver may also serve as a kind of a power source for providing electric power to the sensor assembly.

According to a further example, the sensor assembly includes a signal generator. The signal generator may be implemented as one of a visual signal generator, an acoustic signal generator or a haptic signal generator. By way of the signal generator the sensor assembly may be operable to directly communicate with a user of the injection device. This way, the sensor assembly may provide a confirmation or the like feedback to the user, thus indicating, that e.g. a particular user-induced operation of the injection device has been monitored or has not been monitored correctly. Moreover, the signal generator may indicate to have recognized a gesture of the body part of the user on the sensing surface. The signal generator may be operable to provide and/or to generate visual signals of different color and/or of different and varying durations.

In other examples, the signal generator may be operable to generate an acoustic signal, such as an audible sound. Here, the signal generator may be operable to provide different and distinguishable sound signals, e.g. indicating a successful or unsuccessful detection or measuring of a user-induced action or gesture.

The haptically implemented signal generator may be configured to produce a vibration that is detectable, e.g. palpable by a user.

In further examples, the sensor assembly may include a reminder functionality. Here, the memory of the sensor assembly may be provided with a predefined medication schedule for a particular patient. Accordingly, and when an injection may be due, the processor may prompt the signal generator to generate a user perceivable signal thus reminding the user to conduct or to execute an injection procedure.

In further examples, the sensor assembly includes a position sensor and/or an acceleration sensor. The position sensor may be integrated into the sensor assembly or may be integrated into the injection device. The position sensor may be operably connected with a component of the drive mechanism of the injection device being indicative of a size of a dose currently set or dispensed. The position and/or orientation of the position sensor may be further indicative of an amount of medicament provided in the cartridge. In this way the position sensor may provide quantitative data being indicative of a size of a dose.

In further examples, and when provided with an acceleration sensor the sensor assembly may further detect or measure a particular movement or acceleration of the sensor assembly, e.g. being indicative of a particular gesture conducted by a user when holding the sensor assembly. By way of the acceleration sensor an operation mode of the injection device may be detected accordingly. In some examples, the processor may be operable to generate a control signal and to transmit the control signal to the external electronic device when receiving a characteristic, e.g. a gesture specific, signal from the acceleration sensor.

By way of the transceiver, the sensor assembly may exchange measured data with the external electronic device. The memory of the sensor assembly is typically configured to store use-related data of the injection device. When establishing a communication link with the external electronic device the sensor assembly, hence the memory of the sensor assembly may synchronize with the external electronic device, which device may provide further processing of the data and/or a transmission of the data, e.g. to a healthcare provider.

In some examples, the sensor assembly may be provided with a display, e.g. operable to visually indicate use-specific or user-specific information to a user of the injection device. The display may be implemented into the sensor element. Insofar, the sensor element may be implemented as a touch sensitive display.

Here, the touch sensitive sensor segments may represent an individual pixel of the touch sensitive display. The touch sensitive sensor segments may coincide with the pixels of the touch sensitive display.

The sensor element, and in particular its sensing surface may be provided with a reconfigurable electronic display, e.g. operable to provide visual content to a user of the add-on device or injection device, respectively. Insofar, the sensor element and the sensor assembly provides a twofold function. It is operable to receive input from a user as well as to provide information to the user.

In some examples, the sensor assembly could be operable to provide a visual indication on the sensing surface, such as a number of doses actually set or a number of doses to be set. The sensor assembly could support a patient during setting of a dose as well as during injection of a dose. The sensor assembly, and in particular the touch sensitive display thereof may be operable to provide visual symbols, numbers and/or text in order to guide and/or to assist a user in using of the add-on device or injection device.

The touch sensitive display of the sensor assembly may further provide instructions to the user, such as to set a dose, to inject a dose, to confirm an injection, to submit data between the add-on device and an external electronic device and/or to inform the user when the next injection will be due.

Moreover, the touch sensitive display may be operable to visually indicate a particular portion on the sensing surface that should be touched by the user.

The touch sensitive display may provide static information as well as dynamic information. In some examples, the touch sensitive display of the sensor assembly may be operable to dynamically and visually illustrate a motion of a finger of a user across the sensing surface of the sensor assembly. Here, the user may be even guided of how touch, tip or swipe across the sensing surface of the sensor element.

In another aspect, the present disclosure also relates to an injection device for injecting a dose of a medicament. The injection device includes a body to accommodate a drive mechanism operable to withdraw or to expel the medicament from a medicament container. Typically, the drive mechanism is operable to inject or to expel a dose of the medicament from the medicament container. The injection device further includes at least one of a dose dial and a trigger which is actuatable by a user for injecting and/or for setting of a dose.

The injection device further includes a sensor assembly as described above. The sensor assembly is attached to or is integrated into at least one of the body, the dose dial and the trigger of the injection device. By way of attaching or integrating the sensor assembly to or into at least one of the body, the dose dial and the trigger there may be provided a rather precise and quasi-automated detection, recognition, characterization and measurement of user induced operations of the injection device and/or of gestures executed by a body part of the user.

In addition, the attachment or integration of the sensor assembly to or into the injection device may expand the functionality of the injection device. When the sensor assembly is implemented as a kind of a remote control for an external electronic device, a gesture of a user can be detected and can be transmitted in form of a dedicated or well-defined control signal to the external electronic device. The arrangement or integration of the sensor assembly to at least one of the trigger, the dose dial and the housing of the injection device is beneficial to provide an easy and intuitive accessibility for the user to generate a control or feedback signal for the external electronic device. The control signal and hence the remote control for the external electronic device can be e.g. generated and transmitted by a single finger of a hand which holds the injection device.

The injection device may be implemented as a pen-type injector. It may be implemented as a disposable injection device or as a reusable injection device. In some examples the injection device may include a dial extension, which is subject to a longitudinal and rotative or helical motion during setting of a dose and which is subject at least to a longitudinal sliding movement during dose injection. With other examples the injection device is void of a so-called dial extension. Here, a dose dial and/or trigger may be provided e.g. at a proximal end of the housing of the injection device. The dose member, e.g. in form of a combined dose dial and trigger may be rotatable relative to the body or housing of the injection device for setting of a dose and may be depressible, e.g. by a thumb of a user for injecting the dose.

In other examples, the injection device is implemented as a so-called autoinjector. Here, the user may simply hold the body of the injection device and press the body and hence a distal end of the injection device against a portion of a skin, thereby inducing a rather automated injection procedure, during which an injection needle is urged into the skin and a dose of the medicament is subsequently dispensed or injected.

According to another aspect, the present disclosure also relates to an add-on device configured for fastening to an injection device. The add-on device includes a device body and a fastener for fastening the device body to at least one of a body, a dose dial and a trigger of the injection device. The add-on device includes a sensor assembly as described above. Insofar, the entire functionality of the sensor assembly may be integrated into the add-on device. Typically, and when correctly attached to the injection device the add-on device provides setting and/or injecting of a dose, e.g. by providing or including at least one of an auxiliary dose dial and/or an auxiliary trigger operably engageable with the dose dial and/or with the trigger of the injection device when the add-on device is correctly assembled or attached to the injection device.

Insofar, all features, effects and benefits described above in connection with the sensor assembly equally apply to the injection device and to the add-on device, respectively. In particular when the add-on device is correctly mounted my e.g. to a dose dial of the injection device all effects, features and benefits as described above with regard to the injection device equally apply to the add-on device.

According to yet further aspect, the present disclosure also relates to an injection system including an electronic device and an injection device. The electronic device, which is denoted as an external electronic device is located remote from the injection device. The electronic device may be implemented as a portable electronic device. The electronic device may include one of a smartphone, a smartwatch, a fitness tracker or a tablet computer. In some examples, the electronic device may be implemented as a mobile computer, such as a laptop computer or an immobile computer, such as a desktop computer.

The injection system typically includes an injection device as described above. The external electronic device includes a user interface to communicate with a user of the injection device. The external electronic device further includes a device processor connected to the user interface. The device processor is further operable to provide at least one of an indication and a user instruction to the user via the user interface. The electronic device further includes a device transceiver configured to receive a control signal from the transmitter of the sensor assembly of the injection device.

Here and since the device transceiver of the external electronic device is configured to receive the control signal generated by the sensor assembly of the injection device and transmitted to the electronic device by the transmitter of the sensor assembly the sensor assembly may act as and provide a kind of a remote control for the external electronic device.

Implementation of the sensor assembly of the injection device as a kind of a remote control for the external electronic device is of particular benefit when the sensor assembly is attached or integrated in a component or portion of the injection device that is easily accessible, e.g. by a thumb or by a finger of a hand of a user actually holding the injection device. In this way the external electronic device may provide a user dialog to assist the user in a proper handling or preparing of the injection device. The user may easily reply to requests of the external electronic device with his hand that actually holds the injection device. Specifically, the user may hold the external electronic device in one hand and may hold the injection device in the other hand.

When entering into a dialog with the external electronic device, the user may simply touch the sensing surface of the sensor element provided on the injection device rather than directly interacting with the external electronic device. Providing or implementing the sensor assembly directly on the injection device therefore improves user acceptance and simplifies an assisted use of the injection device.

The injection system typically makes use of the injection device as described above. In some examples the injection system may be implemented by an injection device being void of the above-described sensor assembly. Here, the injection device may be equipped with an add-on device, wherein the add-on device is provided with the sensor assembly as described above. Insofar, all effects, features and benefits as described above in connection with the sensor assembly, the injection device and the add-on device equally apply to the injection system.

Moreover, and since the external electronic device is equipped with a device processor it is also conceivable to transfer at least some or almost all functionalities of the processor of the sensor assembly towards the device processor of the external electronic device. In one example, the processor of the sensor assembly may be limited to simply record the electric touch signal as generated by the touch sensitive sensor segments over time and to transmit the unprocessed or preprocessed electric touch signal as control signals to the external electronic device via the transmitter. A further processing of the electric touch signal(s) or control signal(s) received from the sensor assembly may be then conducted by the device processor of the external electronic device. This way, computational demands for the processor of the sensor assembly may be limited to a minimum, thus allowing to reduce production and manufacturing costs for the sensor assembly.

In effect, and in some examples, it may be the device processor of the external electronic device that is operable to assign a temporal variation of the electric touch signals to one of a plurality of predefined user gestures and to further processed such user gestures. Moreover, it may be the device processor of the external electronic device that is operable to detect, to recognize, to characterize and/or to measure an operation of the injection device on the basis of the temporal variation of the electric touch signals detected by one or more touch sensitive sensor segments of the sensing surface, which electric touch signals are further transmitted from the sensor assembly to the external electronic device via the transmitter.

Insofar, all features, effects and benefits described above in connection with the processor of the sensor assembly may equally apply to the device processor of the external electronic device.

According to still another aspect, the present disclosure further relates to a method of assisting a user to use an injection device by making use of an injection system. The injection system typically includes an injection system as described above. The injection system includes an external electronic device and an injection device. With some examples the injection system includes an external electronic device, an injection device and an add-on device configured for fastening to the injection device. In some examples, at least one of the injection device and the add-on device is provided with a sensor assembly as described above.

The method includes the steps of prompting the user by the user interface of the external electronic device to operate the injection device. For instance, the user interface of the external electronic device may include a display. On the display there may appear an indication to the user prompting the user to set a dose of predefined size.

The user may confirm the indication as provided by the user interface e.g. by tapping or otherwise touching the sensing surface of the sensor element attached to the injection device.

For this, the method includes the step of detecting of a variation of electric touch signals over time caused by the user touching the sensing surface of the sensor element of the sensor assembly of the injection device. In response to the detection of a variation of the electric touch signal there is generated a control signal. Thereafter, the control signal is transmitted to the external electronic device via the transmitter of the sensor assembly. Typically, the control signal e.g. wirelessly transmitted to the external electronic device is received by the transceiver of the external electronic device and is further processed by the device processor of the external electronic device.

In this way, the sensor assembly attached to or integrated into the injection device can be used as a kind of a remote control to e.g. wirelessly transmit a control signal to the external electronic device. This may be of particular use and benefit in situations, where the user already holds the injection device in a palm of one of his hands.

With optional and further method steps it is further conceivable that the external electronic device provides further information or indications to the user after having received the control signal from the sensor assembly. The user may then conduct another or a further device operation and may confirm execution of such a device operation, e.g. by touching the sensing surface of the sensor element of the sensor assembly attached to or integrated into the injection device.

In this way, the external electronic device may provide assistance to the user for conducting individual steps of e.g. setting of a dose and/or or injecting of a dose. The user may confirm or may return control signals or commands to the external electronic device while keeping the injection device in his hand. Since the sensor assembly is provided on a portion of the injection device that is easily accessible by a finger of the hand of the user actually holding the injection device a communication and/or signal exchange or data exchange between the user and the external electronic device can be simplified.

According to a further aspect, the present disclosure also relates to a computer program including computer readable instructions, which when executed by one or numerous processors of an injection system as described above cause e.g. the device processor to prompt the user to operate the injection device via the user interface of the external electronic device, to detect a variation of an electric touch signal over time caused by the user touching the sensing surface of the sensor element of the sensor assembly of the injection device, to generate a control signal on the basis of the temporal variation of the electric touch signal and to transmit a control signal to the external electronic device via a transmitter of the sensor assembly.

Typically, the computer program is configured to conduct the method of assisting a user to use the injection device as described above. The computer program or parts thereof are configured to be executed by the processor of the sensor assembly and/or by the device processor of the external electronic device. Insofar, the computer program may include a first and a second software application, one of which being implemented and executed by the processor of the sensor assembly and the other one of which being implemented and executed by the device processor of the external electronic device.

The present disclosure further discloses and proposes a computer program including computer-executable instructions for performing the method according to the disclosed method/device/system in one or more of the examples enclosed herein when the program is executed on a processor, computer or computer network. Specifically, the computer program may be stored on a computer-readable data carrier. Thus, specifically, one, more than one or even all of the method steps as indicated above may be performed by using a computer or a computer network, typically by using a computer program.

The present disclosure further discloses and proposes a computer program product having program code means, in order to perform the method according to the disclosed method/system in one or more of the embodiments enclosed herein when the program is executed on a computer or computer network. Specifically, the program code means may be stored on a computer-readable data carrier.

Further, the present disclosure discloses and proposes a data carrier having a data structure stored thereon, which, after loading into a processor, computer or computer network, such as into a working memory or main memory of the processor, computer or computer network, may execute the method according to one or more of the examples disclosed herein.

The present disclosure further proposes and discloses a computer program product with program code means stored on a machine-readable carrier, in order to perform the method or parts thereof according to one or more of the examples disclosed herein, when the program is executed on a processor, computer or computer network. As used herein, a computer program product refers to the program as a tradable product. The product may generally exist in an arbitrary format, such as in a paper format, or on a computer-readable data carrier. Specifically, the computer program product may be distributed over a data network.

In another example, the present disclosure proposes and discloses a modulated data signal which contains instructions readable by a processor, a computer system or computer network, for performing the at least parts of a method according to one or more of the examples disclosed herein. Preferably, referring to the computer-implemented aspects of the disclosure, one or more of the method steps or even all of the method steps of the method according to one or more of the examples disclosed herein may be performed by using a processor, a computer or computer network. Thus, generally, any of the method steps including gathering, provision and/or manipulation of data may be performed by using a processor, a computer or computer network. Generally, these method steps may include any of the method steps, typically except for method steps requiring manual work, such as providing the samples and/or certain aspects of performing the actual measurements.

Specifically, the present disclosure further discloses: A computer or computer network including at least one processor, wherein the processor is adapted to perform the method according to one of the examples described in this description, a computer loadable data structure that is adapted to perform the method according to one of the examples described in this description while the data structure is being executed on a processor, a computer, a computer program, wherein the computer program is adapted to perform the method according to one of the embodiments described in this description while the program is being executed on a computer.

Generally, the scope of the present disclosure is defined by the content of the claims. The disclosure is not limited to specific embodiments or examples but includes any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term, “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab')2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not include a full-length antibody polypeptide, but that still includes at least a portion of a full-length antibody polypeptide that is capable of binding to an antigen. Antibody fragments can include a cleaved portion of a full length antibody polypeptide, although the term is not limited to such cleaved fragments. Antibody fragments that are useful in the present invention include, for example, Fab fragments, F(ab')2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates an example of an injection device,

FIG. 2 schematically illustrates an on device configured for fastening to the injection device,

FIG. 3 schematically illustrates a cross-section through an example of an add-on device or an example of a cross-section through a portion of an injection device,

FIG. 4 shows a block diagram of an electronic module for implementing a sensor assembly,

FIG. 5 schematically shows an example of a sensor assembly in a perspective illustration,

FIG. 6 shows the sensor assembly of FIG. 5 is a side view,

FIG. 7 shows an example of the sensing surface slightly touched by a finger of a user

FIG. 8 shows the sensing surface when firmly touched by a finger of the user,

FIG. 9 shows an example of a user when using the injection device equipped with the sensor assembly,

FIG. 10 shows the sensor assembly during a first stage of a dose dialing procedure,

FIG. 11 shows the sender assembly during a second stage of a dose dialing procedure,

FIG. 12 shows an example of an external electronic device in a first stage of assisting a user to use the injection device,

FIG. 13 shows the external electronic device in a second stage of assisting the user,

FIG. 14 shows the external electronic device in a third stage of assisting the user and

FIG. 15 shows the external electronic device in a fourth stage of assisting the user,

FIG. 16 is illustrative of a first gesture conducted by a finger of a user with regards to the sensing surface,

FIG. 17 shows a configuration of the sensing surface during conducting of the castor according to FIG. 16,

FIG. 18 shows a second gesture of a finger of a user with regard to the sensing surface,

FIG. 19 shows the configuration of the sensing surface during the second gesture,

FIG. 20 is illustrative of conducting a third gesture of a user's finger, and

FIG. 21 shows a configuration of the sensing surface during the third gesture,

FIG. 22 schematically illustrates an injection system including an external electronic device and an injection device,

FIG. 23 is illustrative of a block diagram of an example of the external electronic device and

FIG. 24 shows a flowchart of a method of assisting a user to use an injection device.

### DETAILED DESCRIPTION

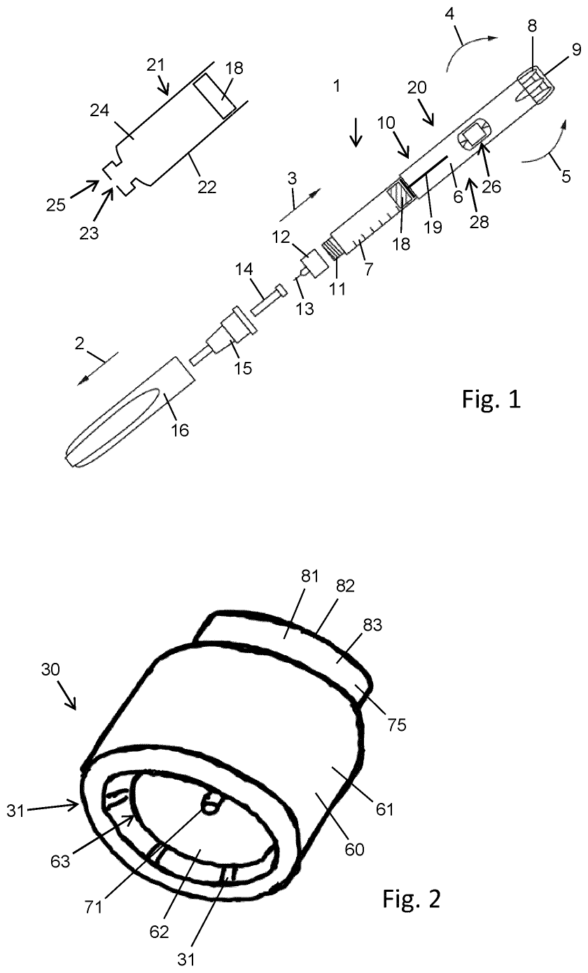

FIG. 1 shows an example of a drug delivery device 1, which is implemented as a handheld injection device. The injection device 1 may include or may be implemented as a pen-type injector. It may be implemented as a disposable injection device or as a reusable injection device. In some examples the injection device 1 is implemented as an autoinjector. The injection device 1 is of elongated shape. It may extend along a longitudinal direction. Towards a longitudinal distal direction 2 the drug delivery device 1 includes a dispensing end for dispensing or injecting the medicament 24. Towards the proximal direction 3 the injection device 1 includes at least one of a dose member 8 and a trigger 9, by way of which a dose of equal or individual or different size can be set and dispensed, respectively.

The injection device 1 includes a housing 10. The housing 10 may include numerous housing components, such as a body 6 and a cartridge holder 7. The body 6 may be sized and configured to accommodate a drive mechanism 20. The cartridge holder 7 is sized and configured to accommodate a medicament container 21, e.g. implemented as a cartridge containing the liquid medicament 24. The medicament container 21 includes a tubular-shaped barrel 22 sealed towards the distal end by a seal 23. The seal 23 may include a pierceable septum fixed to an outlet 25 of the medicament container 21. Towards a proximal end the interior of the barrel 22 is sealed by a piston 18 or stopper, which is slidably disposed inside the barrel 22.

By advancing the piston 18 towards the distal direction 2 a dose of the medicament 24 can be expelled from the medicament container 21. In use the medicament container 21 is arranged inside the cartridge holder 7. The drive mechanism 20 of the injection device 1 includes a piston rod 19, which is displaceable in distal direction 2 for advancing the piston 18 towards the outlet 25 of the medicament container 21. Details of the drive mechanism are not further illustrated and described here. In some examples, the drive mechanism 20 may be implemented as an all-mechanical drive mechanism, where a user has to provide an entirety of a dispensing force required to move the piston rod 19 and hence the piston 18 in distal direction 2. In other examples, the drive mechanism includes a mechanical energy storage configured to provide at least a portion of the dispensing force. Examples of drive mechanism can be found e.g. in WO 2004/078241 A1, WO 2014/033197 A1 or WO 2014/033195 A1 the entirety of which are herein incorporated by reference.

In some examples, as e.g. described or shown in FIG. 9 the injection device 1 and hence the drive mechanism 20 may include a dial extension 27, which projects and moves in proximal direction 3 from a proximal end of the body 6 when or during setting a dose and which returns into its initial distal end position during a dose injection procedure. For this, a user may use a thumb 114 of his hand 110 to exert a distally directed pressure onto the trigger 9 thereby urging the dial extension 27 in distal direction 2 during a dose injection procedure.

For setting or dialing of a dose a user may twist or rotate the dose dial 8, e.g. in a dose incrementing direction 4, hence in a clockwise sense as seen from the proximal end. For correcting a dose previously set the user may also rotate the dose dial 8 in an opposite dose decrementing direction 5. The size of the dose is typically illustrated in a window 26 provided in or on the body 6 of the injection device 1. Prior to inject a dose of the medicament 24 the distal end of the cartridge holder 7 has to be connected with a needle assembly 12. For this, the distal end of the cartridge holder 7 includes a connector 11, e.g. in form of a threaded interface to engage with a complementary shaped threaded counter interface of the needle assembly 12.

The needle assembly 12 is detachably or releasably fixable to the cartridge holder 7. It includes a double-tipped injection needle 13. A proximal end of the injection needle (not shown) is configured to enter into a through opening at the distal end face of the connector 11 or cartridge holder 7 so as to pierce or to penetrate the seal 23 of the medicament container 21. The distal end of the injection needle 13 is typically covered by a detachable inner needle cap 14. The entirety of the needle assembly 12 may be covered by a detachable outer needle cap 15.

The cartridge holder 7 and hence a portion of the housing 10 is to be received in a protective cap 16, which is detachably connectable to the cartridge holder 7 or body 6.

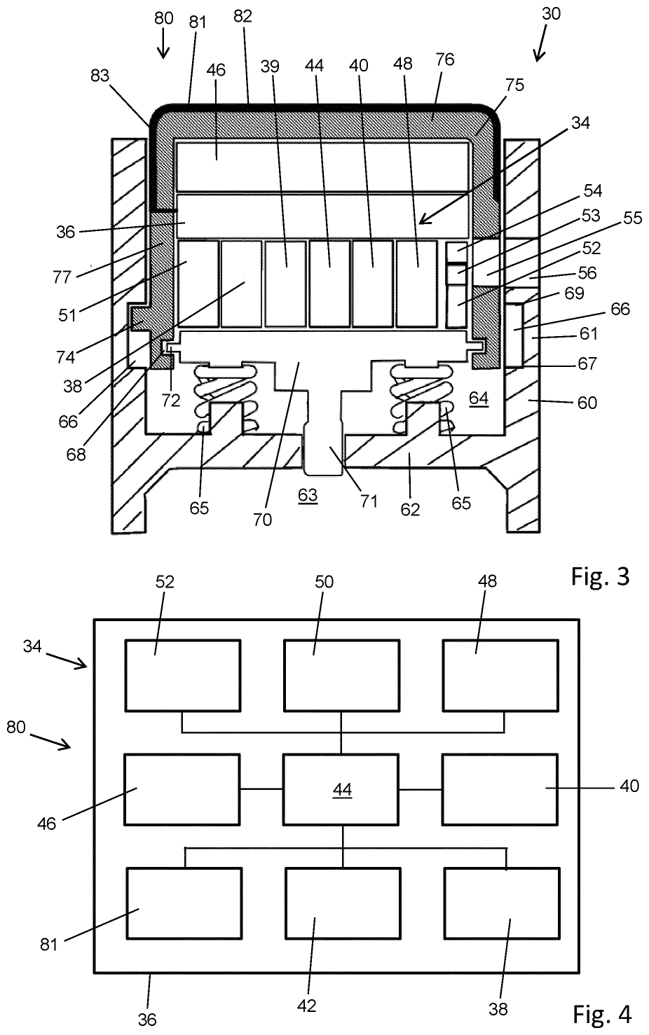

In FIGS. 2 and 3 there is shown an example of an add-on device 30 configured for fastening to the proximal end of the injection device 1. The add-on device 30 includes a sensor assembly 80 with a sensor element 81, which is operable to detect, to recognize, to characterize and/or to measure an operation of the injection device. It may be further operable to detect and/or to characterize a user gesture on a sensing surface 82, 83 of the sensor element 81 of the sensor assembly 80.

The add-on devices 30 as illustrated in FIGS. 2 and 3 is detachably connectable to the dose dial 8. It includes a device body 60 with a tubular-shaped sidewall 61. Towards the distal end the sidewall 61 confines a receptacle 63, which is sized to receive the dose dial 8 and the trigger 9 of the injection device. For this, the inside of the sidewall 60 may include one or numerous fastening ribs 31, which are configured to provide a slip free fastening of the add-on device 32 the dose dial 8.

The receptacle 63 is confined by a radially inwardly extending flange portion 62. The flange portion 62 separates the interior of the device body 60 into a distal receptacle 63 and a proximal receptacle 64. The proximal receptacle 64 is sized to accommodate a support 70 and a cover 75 to house an electronic module 34. The support 70 includes a longitudinally extending stem 71 extending through the flange portion 62 in distal direction. The support 70 is displaceably mounted inside the receptacle 64. It is movable against the action of a return element or several return elements 65 in distal direction 2. The support 70 is connected with a cup-shaped cover 75, which protrudes in proximal direction from the sidewall 61 of the device body 60.

The cover 75 includes a planar-shaped end face 76 facing in proximal direction 3. The end face 76 is provided with the sensor element 81. As illustrated in FIG. 3 the entirety of the end face 76 may be covered by the sensor element 81. In this way, the end face 76 serves as an actuation surface to be depressed by a user, e.g. by a thumb 114 of a user. The user may apply a distally directed force onto the end face 76, thereby urging the cover 75 and the support 70 in distal direction 2 against the action of the return element 65.

Upon releasing the end face 76, the return element 75, e.g. implemented as a return spring, serves to move the support 70 and the cover 75 towards the proximal starting position as illustrated in FIG. 3. At least one of the cover 75 and the support 70 includes a radially outwardly extending projection 74 guided in a longitudinally extending recess 66 on the inside of the sidewall 61 of the device body 60. As seen in longitudinal direction the recess 66 is confined by a proximal stop face 69 and a distal stop face 67. In the initial configuration as illustrated in FIG. 3 the projection 74 is in longitudinal abutment with the proximal stop face 69 of the recess 66.

When depressing the cover 75 in distal direction 2 against the action of the return element 65 a distal end position is reached when the projection 74 engages the distal stop face 67. By way of the projection 74 guided in the groove 66 the longitudinal movement of the support 70 and the cover 75 can be delimited within predefined margins relative to the device body 60.

When depressed in distal direction 2 the support 70 and its stem 71 starts to protrude from the flange portion 62. Since the sidewall 61 is fixable in longitudinal direction to the dose dial 8 the distally directed longitudinal displacement of the support 70 and its stem 71 serves to act on the trigger 9, thereby inducing a distally directed motion of the trigger 9 relative to the dose dial 8 or body 6. The cover 75 is secured to the support 70 with regard to the longitudinal direction. It may be freely rotatable relative to the support 70, in particular during a dose injection procedure. During dose injection the dose dial 8 may be subject to a rotation in the dose decrementing direction 5 while the trigger 9 remains rotationally locked to the dial extension 27 and/or to the body 6. Here, the support 70 and/or the cover 75 may form or constitute an auxiliary trigger of the add-on device.

Here, the inside of a sidewall of the cover 75 may include at least one of a radial protrusion and a recess 68 to engage with a complementary-shaped radial recess or protrusion 72 of the support 70. The cup-shaped cover 75 connected or fastened to the support 70 provides a receptacle for an electronic module 34 arranged inside the hollow space formed by the support 70 and the cover 75.

The sensor assembly 80 as described herein may be somewhat identical or equivalent to the electronic module 34; and vice versa. The electronic module 34 may include a printed circuit board 36. It may further include a transceiver 38, 39, a memory 40, a clock 42, a processor 44, a power source 46, an acceleration sensor 48, a position sensor 50, signal generator 51, 52 and e.g. light sources 53, 54. It may also include a microphone.

Moreover, a sidewall 77 of the cover 75 may include a window 55 aligned with a respective window 56 in the sidewall 61 of the device body 60. This way, visual signals producible by different light sources 53, 54 located inside the hollow space of the cover 75 can be perceived and visually detected from outside the add-on device 30.

The two light sources 53, 54 may belong to a visual signal generator 52, that is operable to produce or to generate visual signals of different color and/or of variable duration. The windows 55, 56 may be provided with a light pipe or with a light guiding structure. This way, the device body 60 can be protected against ingress of dust or humidity.

In FIG. 4 there is shown a block diagram of one example of the electronic module 34 and/or of the sensor assembly 80. As illustrated also in FIG. 3 the processor 44 is mounted on the printed circuit board 36, which is connected to the sensor element 81 of the sensor assembly 80 substantially covering the end face 76 of the cover 65.

In general, the electronic module 34 can be mounted on the printed circuit board 36. The electronic module 34 may be configured to communicate with an external electronic device 100, as e.g. illustrated in FIG. 28. The external electronic device 100 may be implemented as a mobile electronic device. It may include a smartwatch, a smart phone or a tablet computer. The electronic module 34 includes a transceiver 38, which is configured to establish or to built-up a communication link between the external electronic device 100 and the electronic module 34. A respective communication link can be realized in a wireless or wired manner.

The electronic module 34 may be configured to exchange data with the external electronic device 100. Data being indicative of an operation of the injection device and gathered or collected by the sensor assembly 80 may be transmitted to the external electronic device via the transceiver 38. The transceiver 38 may be implemented as a Bluetooth transceiver or as a BLE transceiver. The further transceiver 39 may be implemented as a NFC transceiver. The two transceivers may distinguish with regard to their communication protocol and/or with regard to their spatial range.

The electronic module 34 and hence the sensor assembly 80 includes a memory 40 configured to store a plurality of measurement results of the sensor assembly 80. The electronic module 34 and hence the sensor assembly 80 further includes a clock 42 configured to provide each of a plurality of measurement data of the sensor assembly 80 with a time index indicating the point of time and/or date of the detection of the respective operation of the injection device 1.

The processor 44 of the sensor assembly 80 and hence of the electronic module 34 is configured to control operation of the sensor assembly 80 and hence operation of the sensor element 81. The sensor assembly 80 and hence the electronic module 34 is further equipped with a power source 46 configured to power the processor 44 and the sensor assembly 80. The power source 46 may be implemented as a battery. The sensor assembly 80 may further include an acceleration sensor 48 configured to detect an injection operation of the injection device 1 and/or to detect or to classify gestures of a user when using the sensor assembly 80 and/or the injection device 1. For example, and when implemented as an autoinjector, the acceleration sensor may detect an acceleration of a needle of the autoinjector upon executing a dispensing or injection procedure.

The sensor assembly 80 may further include a position sensor 50, which may be operable to detect the position or orientation of a dedicated component of the drive mechanism 20 of the injection device 1. The position sensor may be operable to detect e.g. the position of a last dose nut, of a piston rod 19 or the like component of the drive mechanism 20 being indicative of the amount of medicament located inside the medicament container 21.

Moreover, the sensor assembly 80 or electronic module 34 includes a signal generator 52, which may be implemented as a visual signal indicator including at least one light source 53, 54. Additionally or alternatively, the signal generator 51 may be implemented as a haptic signal generator, e.g. configured to generate a perceivable vibration of the electronic module 34. In further examples, the signal generator 51 may include an audible signal generator, configured to generate an audible sound.

In addition and in further examples, the electronic module 34 and/or the sensor assembly 80 may include a microphone, by way of which characteristic click noises of the injection device can be detected and processed, thereby allowing to derive or to measure a size of a dose currently set or injected by the injection device 1.

The presently illustrated implementation of the electronic module 34 in an add-on device 30 is only exemplary. Especially with reusable devices it is also conceivable that the entire functionality of the add-on device 30 and hence the entire functionality of the sensor assembly 80 and the electronic module 34 is implemented in an injection device 1. Here, the receptacle 64, which is sized to accommodate the hardware components of the electronic module 34 may be provided inside the dose dial 8, which is covered or closed in proximal direction by the trigger 9. Here, the trigger 9 of the injection device replaces the cover 75 and the support 70. The stem 71 of the support 70 and hence of the trigger 9 may be then operably engaged with the drive mechanism in order to induce or to trigger a dispensing operation thereof. The device body 60 may be provided or represented by the tubular shaped those the dial 8.

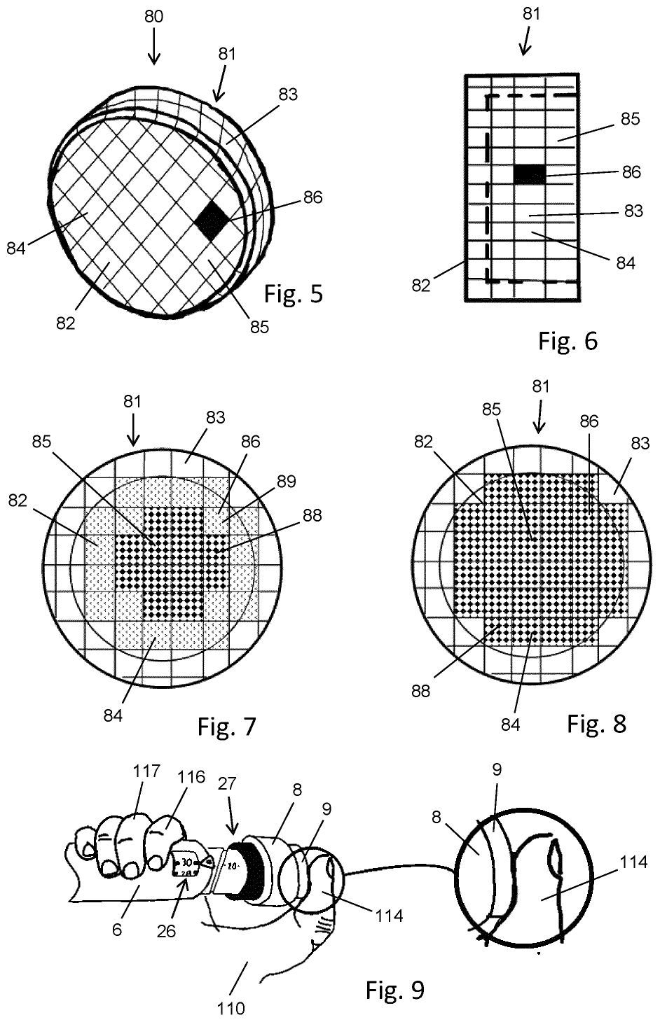

In FIGS. 2, 3 and 5-8 one example of a sensor assembly 80 is schematically illustrated. The sensor assembly 80 includes a sensor element 81 with numerous touch sensitive or pressure sensitive sensor segments 84, 85, 86 on a sensing surface 82, 83. The sensor assembly 80 may include a regular arrangement of touch sensitive sensor segments 84, 85, 86 as illustrated in FIGS. 5 and 6. The individual sensor segments may be comparable to pixels of a touch sensitive display. The sensor segments 84, 85, 86 may each include a capacity measuring device by way of which a mechanical contact with a body part of a user can be precisely detected.

In some examples, the sensor segments 84, 85, 86 are pressure sensitive. They are hence operable to generate or to modify an electric touch signal when touched by a body part of a user. The sensor segments 84, 85, 86 may be spatially distributed across the sensing surface 82, 83 of the sensor element 81. The sensor element 81 may provide a spatially resolved detection of a sensing area 88, 89 that is actually touched by or which is in mechanical contact with the skin of a user.

In some examples, the individual sensor segments 84, 85, 86 are not only touch sensitive but are also operable to generate electric touch signals being indicative of a pressure or strength of the mechanical contact with the body part of the user. Hence, the touch sensitive sensor segments 84, 85, 86 are operable to generate different electric touch signals being indicative of the size of a pressure or force applied to the respective sensor segment.

Insofar, the touch sensitive sensor segments 84, 85, 86 are operable to generate electric touch signals that differ in at least one of a magnitude, an amplitude, a sign or frequency. Those differences in the electric touch signals are detectable by the processor 44 of the sensor assembly 80 and can be evaluated so as to detect, to recognize, to characterize and/or to measure at least one of a user-induced operation of the injection device 1 and a gesture conducted by a body part of the user.

In the example as shown in FIGS. 5-8, the sensor element 81 includes a planar-shaped sensing surface 82 of circular shape and a tubular shaped sensing surface 83, which is located longitudinally adjacent to the outside circumference of the sensing surface 82. Insofar, the sensor element 81 includes a cup-shaped receptacle, which is sized e.g. to receive the trigger 9 of the injection device 1 and/or the cover 75 of the add-on device 30.

In further examples, it is conceivable that the cup-shaped sensor element 81 is configured to receive a dosing element, which combines the functionality of a dose dial 8 and a trigger 9 in a single part.

With the illustration of FIGS. 5 and 6, only one sensor segment 86 of the plurality of sensor segments 84, 85, 86 is actually active and detects a contact with a body part of a user.

in some examples, the sensor element 81 only includes a planar-shaped sensing surface 82 fastened to the proximal end face of a trigger 9 or of a cover 75 of an add-on device 30. With other examples the sensor element 81 may exclusively include a tubular shaped sensing surface 83 configured to confine a tubular-shaped member 28 of the injection device 1. The tubular shaped member 28 may be provided by the tubular shaped dose dial 8 or by the tubular shaped body 6 of the injection device 1.

In FIG. 9, a typical scenario of use of the injection device 1 provided with a dial extension 27 is illustrated. For setting of a dose the user rotates the dose dial 8 relative to the body 6. The dose dial 8 is a part of the dial extension 27, which then starts to project in proximal direction 3 from the proximal end of the body 6 as a dose of increasing size is set or dialed.

As a consequence, an increasing number of dose indicating indicia shows up in the dose indicating window 26. For injecting of a dose the user uses his hand 110 and clasps the body 6 of the injection device with his palm 112 thereby using at least two fingers 116, 117 to hold the body 6 of the injection device 1. The user may then use his thumb 114 to induce a dispensing action by depressing the trigger 9 in distal direction 2.

Since the trigger 9 and hence the entire dial extension 27 is subject to a distally directed longitudinal displacement relative to the body 6 during dose injection the angle at which the thumb 114 is oriented relative to the trigger 9 will be subject to a gradual change. Hence, during dispensing of the dose the thumb 114 is subject to a milling motion or natural rolling motion, which is detectable by the spatially resolved touch sensitive sensing surface 82 of the sensor element 81 covering the proximal face of the trigger 9 or of the cover 75 of the add-on device 30.

Moreover and as it is illustrated in greater detail in FIGS. 7 and 8 the touch sensitive sensor segments 84, 85, 86 are operable to generate different electric touch signals dependent on the force level or pressure level applied to the respective sensor segments 84, 85, 86. In FIG. 8a rather high or a comparatively high pressure level is indicated by comparatively dark shaded sensor segments 84, 85, 86. A comparatively low pressure level is indicated in FIG. 7 by relatively weak or brightly shaded sensor segments 84, 86, while the sensor segment 85 is subject to a comparatively high pressure level.

In this way there is not only provided a spatially resolved contact profile across the one-or two-dimensional sensing surface 82, 83 of the sensor element 81 but the pressure profile may be also indicative of different pressure levels applied across the sensing surface(s) 82, 83.

With the example of FIG. 7, there are altogether only 12 sensor segments on the sensing surface 82 that are subject to a comparatively large force or pressure effect. Here, only the exemplary sensor segment 85 is subject to a comparatively high pressure. The sensor segments 84, 86 are located radially outside the sensing area 88 of high-pressure. They are subject to a moderate or comparatively low pressure.

The sensor segments 84, 86, which are subject to a comparatively low or moderate pressure form or constitute a sensing area 89 of low or moderate pressure. Those sensor segments 85 that are subject to a comparatively high pressure form or constitute a sensing area 88.

As illustrated in FIG. 8, the sensing area 88 is located almost in the middle of the sensing surface 82. It is surrounded by the sensing area 89 of comparatively low pressure. This example may represent a case, where a user applies a moderate pressure onto the sensing surface 82, e.g. with his thumb 114. Since the thumb 114 is of convex shape and is of a certain elasticity by increasing the pressure the thumb 114 of the user will be subject to a respective deformation, thus increasing the lateral extent of the sensing area 88 of high-pressure. As indicated in FIG. 8 those sensor segments 84, 86, which were previously only subject to a comparatively low pressure, as shown in FIG. 7, are now subject to a comparatively high pressure.

A change of the pressure profile from the example of FIG. 7 towards the example of FIG. 8 can be precisely detected and monitored by the processor 44, which is configured to individually process the electric touch signals produced or modified by each of the sensor segments 84, 85, 86. Insofar, the processor 44 is configured to detect or to recognize an increase or modification of the sensing area 88.

Generally, it should be noted that the sensor segments 84, 85, 86 specifically mentioned herein exemplary represent the entirety of the sensor segments provided on the sensing surfaces 82, 83. The spatial resolution of the sensor assembly 18 may depend on the total number of sensor segments 84, 85, 86 and their arrangement on the sensing surfaces 82, 83.

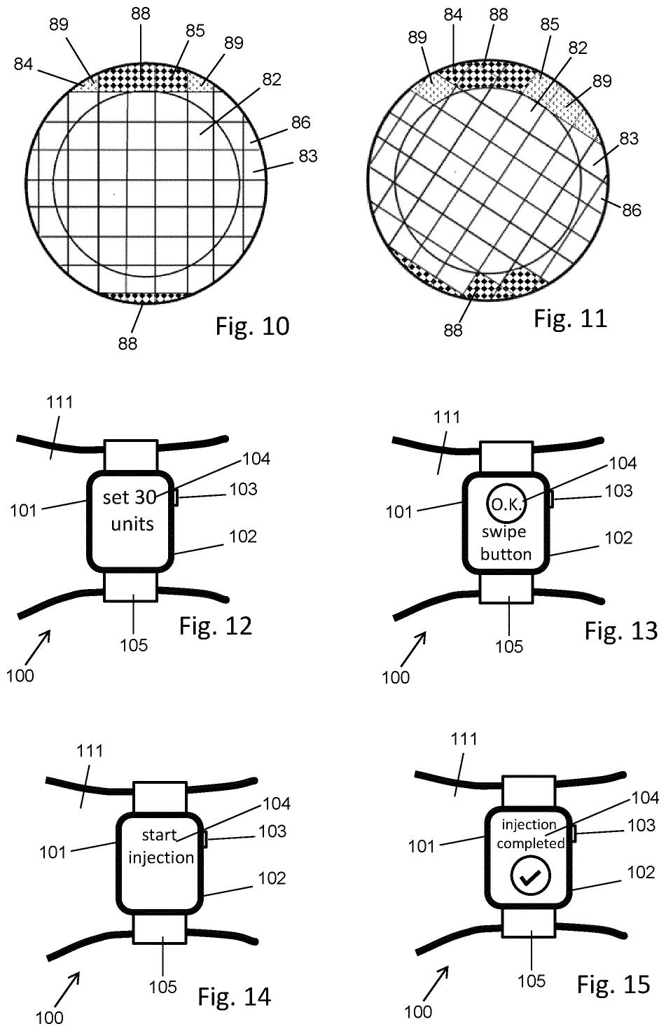

As particularly illustrated in FIGS. 9 to 11 in a further configuration the user may use a thumb 114 and the index finger 116 to rotate a dose dial 8, which may be likewise provided with the tubular shaped sensing surface 83 of the sensor element 81. In FIG. 10 it is only the sensor segment 85 which is subject to a substantially high-pressure whereas the sensor segment 84 is subject to a lower or moderate pressure and the sensor segment 86 is not subject to any pressure at all. By rotating the dose dial 8, e.g. clockwise a pressure profile along or across the sensing surface 83 is subject to a gradual change. In the configuration of FIG. 10 the sensing areas 88, which coincide with the position of the thumb 114 and the index finger 116 are circumferentially framed or enclosed by sensing areas 89 of rather low pressure or force.

By rotating the dose dial 8 the sensor segment 84 is exposed to an increase of the contact pressure. As illustrated, the sensing areas 88, 89 move in circumferential direction with regard to the position of the sensor segment 84, 85, 86. This movement of the sensing areas 88, 89 can be detected and tracked as well as quantitatively measured by the processor 44. In this way, the sensing surface 83 and the individual sensor segments 84, 85, 86 also allow and support a quantitative measurement of a degree of rotation of the dose dial 8 relative to the body 6.

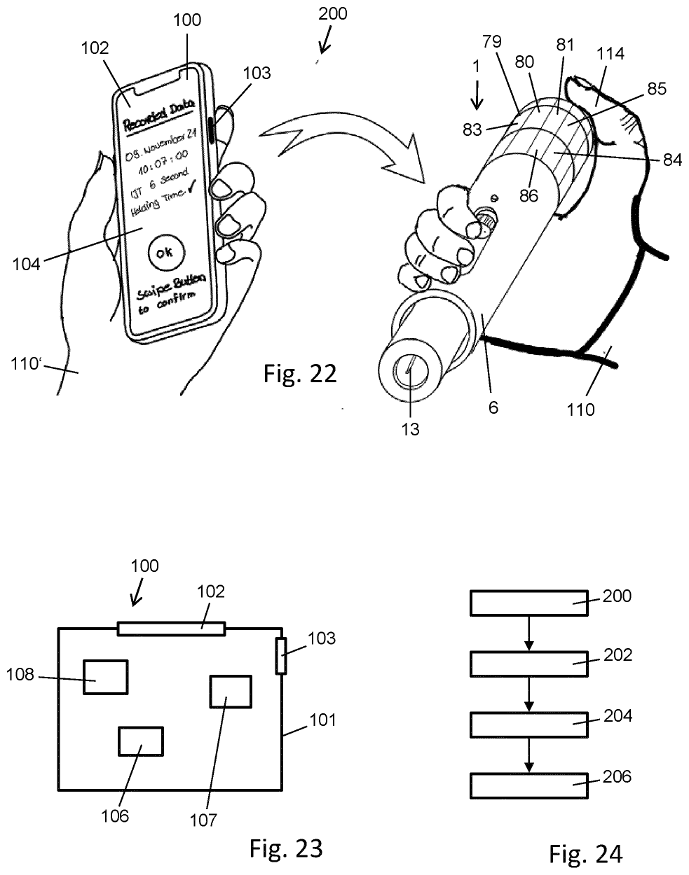

In further examples, e.g. illustrated in FIG. 22 the sensor assembly 80 includes a flexible sheet 79 configured to clasp around or to wrap around the body 6 of an injection device 1. There, the injection device 1 may be implemented as an autoinjector. The injection device 1 may be void of a separate trigger to be depressed e.g. by a thumb 114 of a user. Rather, a dose dispensing or dose injection operation may be simply triggered by bringing a distal dispensing end of the injection device 1 into contact with the skin and by applying a moderate pressure onto the skin via the housing 10 or body 6 of the injection device 1.

Then, the injection device 1 may automatically start a dispensing procedure by pushing the injection needle into the skin followed by a delivery or injection of the medicament into the skin portion which is actually punctured.

Here, and in a typical scenario of use and before applying a distally directed pressure onto the body 6 of the injection device a user may clasp around the body 6 of the injection device 1, which is provided with the sensor assembly 80 as described before. The sensor assembly 80 may include a flexible sheet 79 or foil with numerous sensor segments 84, 85, 86 allowing for a precise and spatially resolved recording and detection of a holding force or pressure applied by the fingers 114, 116, 117 and the palm 112 of the hand 110 of the user.

When a user simply holds the injection device 1 in his hand 110 the contact area between the palm 112 and the sensor element 81 may be detected as a sensing area 88. When the user brings the dispensing end of the injection device 1 in contact with the skin and starts to urge the injection device 1 against the skin a slight but measurable deformation in the contact area between the hand 110 and the sensor element 81 will arise. Since the hand 110 flexes along the longitudinal direction on the sensing surface 82 towards the distal end the sensor assembly 80 is operable to record a respective movement of the sensing areas 88. The temporal variation and/or movement of the sensing areas 88, 89 can be detected and/or recorded by the processor 44.

Moreover, and at the end of a dose dispensing procedure the processor 44 may be further operable to measure a duration during which a sensing areas 88 remains substantially constant. In the same way, the sensor assembly 80 may be configured to detect how long the user keeps the thumb 114 firmly pressed on the trigger 9, e.g. at the end of a dose dispensing procedure. In this way, the sensor assembly 80 is configured to automatically detect and to record a prescribed holding time, during which the injection needle 13 should remain in the punctured tissue after termination of the dose injection of the medicament.

Upon termination of a dose injection procedure the sensor, assembly 80 and/or electronic module 34 may be configured to provide a perceivable feedback to the user, either visually, acoustically or in a palpable way. For this, the onboard signal generators 51, 52 can be used or activated by the processor 44. Alternatively, and when the sensor assembly 80 is in a communication mode with the external electronic device 100 a respective feedback signal may be also generated by the external electronic device 100.

When the end of an injection procedure is reached, the user may be obliged to maintain a pressure on the trigger 9. This may lead to rather constant electric touch signals provided by the numerous touch sensitive sensor segments 84, 85, 86. Insofar, a rather constant and non-modifying pressure profile as measured by the sensor assembly 80 and simply followed by a complete release of the sensing surface 82, wherein the body part is disconnected from and is hence no longer in contact with the sensing surface 82, 83, is a direct indication that the user complied with the prescribed holding of the injection device after termination of the dose injection procedure.

Here, the sensor assembly is configured to automatically distinguish between different operation modes of the injection device. It may automatically record the time and/or the duration and which such different operations of the injection device took place.

In this way, the precision and quality of the data gathered by the sensor assembly can be improved.

In FIGS. 12-15, 22 and 23, numerous examples of external electronic devices 100 are schematically illustrated. The external electronic device 100 may be implemented as a smartwatch or smartphone. It includes a housing 101 and a user interface 102, which may be implemented as a display or as touch sensitive display. The external device 100 may further include a control element 103 operable or actuatable by a user of the device. By way of a wristband 115 the external electronic device 100 can be attached to a wrist 111 of the user. On the display 102 there may be provided numerous visual items 104, in form of symbols, text or the like information by way of which the user may be assisted in using the injection device 1.

With a communication link between the sensor assembly 80 and the electronic module 34 it may be no longer necessary that the user confirms a certain operation mode of the injection device 1 manually with the external electronic device 100. Due to a communication link, e.g. a wireless communication link between the external electronic device 100 and the sensor assembly 80 the external electronic device 100 may be automatically provided with information such as setting of a dose at a certain point of time and dispensing of a dose at a certain point of time.

Moreover, the sensor assembly 80 may be further configured to detect or to quantitatively measure a size of a dose actually set by a user of the injection device. Upon detecting a dispensing procedure and by distinguishing a dispensing procedure from e.g. a dose setting procedure the sensor assembly 80 and the electronic module 34 may automatically provide the relevant information to the external electronic device 100, which may provide further data processing or data analysis. Moreover, the external electronic device 100 may be configured to transmit the acquired data to a healthcare provider for further data analysis and for controlling the user's compliance with a prescribed medication schedule.

The user interface 102 may include at least one of a display, e.g. a touch sensitive display, an audible and/or a haptic or palpable signal generator by way of which the external electronic device 100 may communicate with the user of the injection device 1. The external electronic device 100 further includes a device processor 106, a device memory 107 and a device transceiver 108. The device transceiver 108 may be implemented as a wireless transceiver. It may establish a communication link with the transceiver or transmitter 38, 39 of the sensor assembly 80.

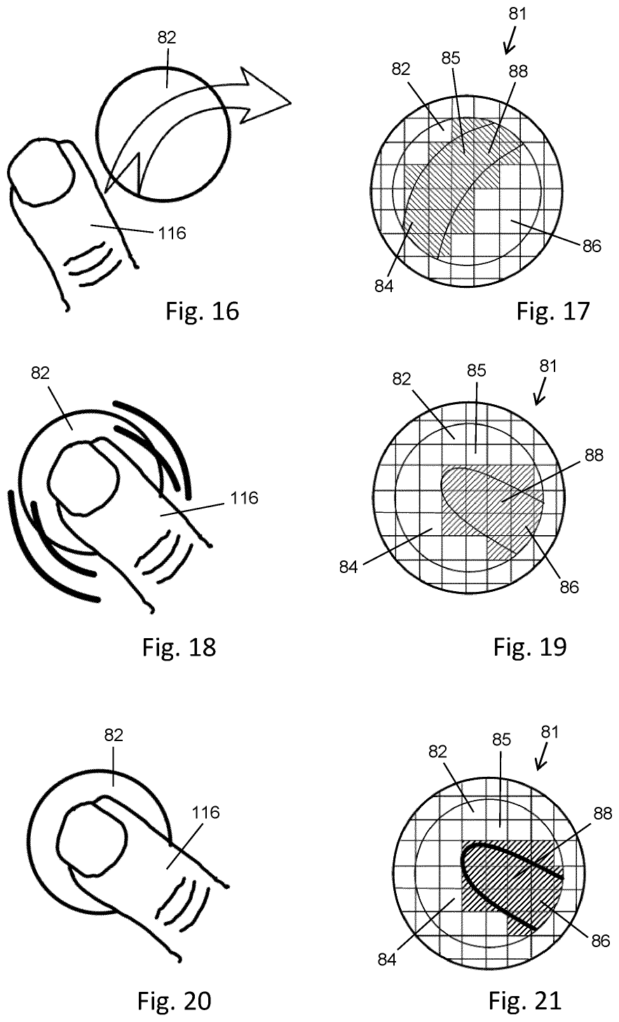

In FIGS. 16-21, numerous gestures conducted by a user of the injection device 1 are schematically illustrated. In FIG. 16, a user uses his index finger 116 to slide over the sensing surface 82 of the sensor element 81. The sliding motion as indicated by a bended arrow. In FIG. 16, there is illustrated a corresponding configuration of the sensing surface 82 when integrated over time while the finger 116 swipes over the sensing surface 82. The touch sensitive sensor segments 84, 85 that are located on the path followed by the index finger 116 while swiping over the sensing surface 82 are indicated by a shaded design. Those touch sensitive sensor segments 86 that are not touched by the finger 116 remain in white color.

The sliding motion of the index finger can be dynamically tracked and sensed over time by processing and/or evaluating the electric touch signals generated by the individual sensor segments 84, 85 that are touched by the finger 116 when the finger 115 swipes over the sensing surface 82.

The processor 44 of the sensor assembly 80 is configured to detect and to process the individual electric touch signals generated by those sensor segments 84, 85 that lie within the sensing area 88 as indicated in FIG. 17. Here, processor 44 is operable to derive a spatial and/or temporal profile of the electric touch signals generated by numerous touch sensitive sensor segments 84, 85, 86. Accordingly, the processor 44 is operable to recognize the swiping motion of the finger 116.

In FIG. 18, another gesture is indicated, where a finger 116 conducts multiple short-term tapping motions on the sensing surface 82. Here, the sensing area 88 following the contour of the finger 116 is visibly marked on the illustration of the sensing surface 82 according to FIG. 19. The sensor segment 86 is located within the sensing area 88 whereas the sensor segments 84, 85 are located outside the sensing area 88. The processor 44 is configured to detect a single as well as multiple short-term tapping motions of the body part or finger 116 on the sensing surface 82.

With the further example as illustrated in FIGS. 20 and 21 the finger 116 and hence the body part of the user applies a pressure of a particular strength onto the sensing surface 82 of the sensor element 81. Accordingly and as indicated by the dark shaded sensing area 88 and the dark shaded touch sensitive sensor segment 86 the processor may be provided with sensing signals that are indicative of a comparatively high pressure applied by the body part or finger 116. Typically, the touch sensitive sensor segments 84, 85, 86 are sensitive to the pressure applied and may be operable to modify or to generate distinguishable electric touch signals being indicative of the magnitude of the pressure currently applied.

The processor 44 may be operable to distinguish between electric touch signals being indicative of a pressure above a predefined threshold. Insofar, with all examples as illustrated in FIG. 16-21 the processor 44 is operable to distinguish between these characteristic user gestures.

In some examples a swiping motion or swiping gesture as illustrated in FIG. 16 may be used to generate a control signal and to transmit the control signal from the sensor assembly to the external electronic device 100. When received by the external electronic device 100, the control signal generated on the basis of the swiping gesture may be interpreted as a confirmation, e.g. by the device processor 106.

A multiple short-term tapping as illustrated in FIG. 18 may be used for the generation and transmission of another control signal to the external electronic device 100, which may be interpreted as a delete or reset signal, e.g. in a dialog with a user. The further gesture as illustrated in FIG. 20, which may include a long lasting and/or a high-pressure gesture may cause the processor of the sensor assembly to generate a further control signal and to transmit the further control signal to the external electronic device, thereby indicating that the injection device is currently used for injecting of a dose of the medicament.

With an injection system 200 as illustrated in FIG. 22, a user may hold the external electronic device 100 in a left-hand 110′ and may hold the injection device 1 in a right-hand 110. The external electronic device 100 may provide an indication or an instruction to the user in form of a visual item 104 on the display 102 prompting the user to confirm e.g. the recording of a dose dispensing procedure. As indicated on the display 102 the user is prompted to swipe the sensing surface 82, 83 of the sensor element 81, which is attached to or integrated in the injection device 1.

The user may e.g. use his thumb 114 to conduct a respective swiping motion. The swiping motion causes the touch sensitive sensor segments 84, 85, 86 of the sensor element 81 to generate or to modify an electric touch signal, which is processed by the processor 44. The processor 44 may process the electric touch signal(s) and may recognize a particular gesture of the respective body part of the user and may generate a control signal on the basis of such a gesture recognition.

The processor 44 may then transmit the respective control signal to the external electronic device 100. In this way, the sensor assembly 80 attached or attachable to the injection device 1 provides an effective remote control for the external electronic device 100. It serves to enhance and to facilitate to assist a user in using an injection device 1 while instructed by a software application provided or executed by the external electronic device 100.

In FIGS. 12-15, there is illustrated an exemplary scenario of a method of assisting a user to use the injection device 1 by making use of an injection system 200 as described herein. The external electronic device 100 is e.g. implemented as a smartwatch worn on a wrist 111 of a hand 110 of a user. The user may wear the electronic device 100 e.g. on his left hand 110′ while keeping the injection device 1 in his right hand 110. The electronic device 100 may be configured to execute user assisting instructions, e.g. to execute a software application that assists a user to correctly use the injection device 1. In an initial configuration as illustrated in FIG. 12 the user interface 102, e.g. implemented as a display, may indicate to the user that setting of a dose of e.g. 30 units is currently due.

The user may then use the injection device 1 and set or dial a dose of required size. For this, the user may e.g. dial the dose dial 8 in a clockwise direction. During setting of the dose the sensor assembly 80 may detect the dialing motion e.g. by a temporal variation of signals from sensor segments 84, 85, 86 e.g. provided on the tubular sensing surface 83 attached to the outside circumference of the dose dial 8. Here and as described in connection with FIGS. 10 and 11 the sensor assembly 80 may be even configured to quantitatively determine or to quantitatively measure the size of the dose currently set. Upon detecting or determining that the dose of required size has been set the processor 44 of the sensor assembly 80 may be operable to generate a respective control signal and transmit the same to the external electronic device 100.

When the sensor assembly 80 has detected that a dose of required size has been set, e.g. when the external electronic device 100 receives a respective control signal from the sensor assembly 80, the user interface 102 may switch to the configuration as illustrated in FIG. 13. Here, the user may be prompted to confirm setting of the dose. He may be prompted to confirm the dose setting by swiping a particular portion, e.g. the planar-shaped sensing surface 82 of the sensor assembly 80. This swiping motion and hence the confirmation that the dose of required size has been actually set can be conducted by a finger 114, 116, 117 of the user's right hand 110.

Independent of a quantitative measurement and when having set the dose of required size the user may confirm setting of the dose, e.g. by conducting a swiping motion across at least one of the sensing surfaces 82, 83. The swiping motion is typically recorded or detected by the sensor assembly 80, which in response to such detection generates a further control signal and transmits the same to the external electronic device 100.

After having confirmed setting of the dose by conducting a gesture with regard to the sensor assembly 80 and/or upon receiving the further control signal the user interface 102 may switch into a configuration as illustrated in FIG. 14. Here, the user may be prompted to start the injection procedure. He may then apply a distally directed pressure onto the trigger 9. Alternatively, and with an autoinjector the user may urge the body 6 against a portion of the skin. Applying pressure onto the trigger 9 as well as urging the body 6 against the portion of the skin can be qualitatively as well as quantitatively detected by the sensor assembly 80. Dispensing or injecting of a dose may be accompanied by a gesture as described in connection with FIGS. 20 and 21. The sensor assembly may record application of a pressure above a first and/or a second threshold over a time interval of a characteristic duration.

If the trigger 9 is subject to a gradual longitudinal displacement, during the process of dose injection there may be recordable a milling or natural rolling motion of e.g. a thumb 114 of the user relative to the planar shaped sensing surface 82 covering the trigger 9. This particularly applies, where the trigger 9 is provided at the proximal end of a dial extension 27 of an injection device 1. The rolling motion of the thumb 114 may be detectable and/or recordable by the sensor assembly 80. Here, the sensing area 88 may be subject to a temporal variation, e.g. it may be subject to a movement and/or to a measurable geometric variation. At the end of an injection procedure the user may have to keep the injection needle 13 in the skin for a predetermined time interval, also denoted as the holding time. During this time interval the sensor assembly 80 may only record a rather constant and hence non-varying sensing area 88. By analyzing the touch signals generated by individual sensor segments 84, 85, 86 the beginning and termination of the injection procedure as well as the beginning and termination of a holding procedure can be precisely detected.

After termination of the injection procedure and/or after lapse of the holding time the user may remove the thumb 114 from the trigger 9. This removal is immediately recorded by the sensor assembly 80, which is operable to generate a respective control signal and transmit such a control signal to the electronic device 100. Accordingly, the electronic device 100 may provide an indication, e.g. in form of a visual item 104 to the user that the injection procedure has been completed. Injection-related data recorded during the process of dose setting and/or dose injecting may be summarized on the user interface 102 as e.g. illustrated in FIG. 22. The user may then finally confirm to store the acquired data locally, e.g. in the device memory 107 and/or to transmit the acquired data to e.g. a healthcare provider. This confirmation may require another swiping motion as e.g. illustrated in FIG. 16 or 17. Alternatively and if the user would like to reject or to disapprove storing and/or transmission of data he may conduct a double-tapping as illustrated in FIGS. 18 and 19.

As further illustrated in the flowchart of FIG. 24, the method of assisting the user in using the injection device includes a step 200 of prompting the user via the user interface 102 of the external electronic device 100 to operate the injection device 1 as e.g. illustrated in FIG. 12. In the subsequent step 202 a variation of electric touch signals over time is detected that are caused by the user touching the sensing surface 82, 83 of the sensor element 81 of the sensor assembly 80, typically, when the sensor assembly 18 is attached or fixed to the injection device 1. In the proceeding step 204 the sensor assembly 80 generates a control signal on the basis of the temporal variation of the electric touch signal and in step 206 the touch control signal is transmitted to the external electronic device via the transmitter or transceiver 38, 39 of the sensor assembly 80. Insofar, the sensor assembly provides a remote control for the external electronic device and helps the user to enter different user comments into the electronic device 100 without the requirement of directly entering a command in the electronic device 100 by tapping on the device 100.

### REFERENCE NUMBERS

1 injection device
2 distal direction
3 proximal direction
4 dose incremention direction
5 dose decrementing direction
6 body
7 cartridge holder
8 dose dial
9 trigger
10 housing
11 connector
12 needle assembly
13 injection needle
14 inner needle cap
15 outer needle cap
16 protective cap
18 piston
19 piston rod
20 drive mechanism
21 medicament container
22 barrel
23 seal
24 medicament
25 outlet
26 window
27 dial extension
28 tubular member
30 add-on device
34 electronic module
36 printed circuit board
38 transceiver
39 transceiver
40 memory
42 clock
44 processor
46 power source
48 acceleration sensor
50 position sensor
51 signal generator
52 signal generator
53 light source
54 light source
55 window
56 window
60 device body
61 sidewall
62 flange portion
63 receptacle
64 receptacle
65 return element
66 recess
67 stop face
68 recess
69 stop face
70 support
71 stem
72 projection
74 projection
75 cover
76 end face
77 sidewall
79 sheet
80 sensor assembly
81 sensor element
82 sensing surface
83 sensing surface
84 sensor segment
85 sensor segment
86 sensor segment
88 sensing area
89 sensing area
100 external device
101 housing
102 user interface
103 control element
104 visual item
105 wristband
106 device processor
107 device memory
108 device transceiver
110 hand
111 wrist
112 palm
114 thumb
116 finger
117 finger
1-19. (canceled)

20. A sensor assembly for an injection device, the sensor assembly comprising:
a sensor element attachable to the injection device and comprising a sensing surface, the sensing surface comprising a touch sensitive sensor segment, the touch sensitive sensor segment being operable to generate or to modify an electric touch signal when touched by a body part of a user,
a processor connected to the sensor element, operable to detect a variation of the electric touch signal over time and to generate a control signal on the basis of the temporal variation of the electric touch signal, and
a transmitter connected to the processor and operable to transmit the control signal to an external electronic device.

21. The sensor assembly according to claim 20, wherein the sensing surface comprises a number of touch sensitive sensor segments each of which being operable to generate or to modify an electric touch signal when touched by a body part of a user.

22. The sensor assembly according to claim 20, wherein the processor is operable to generate the control signal on the basis of the temporal variation of a plurality of electric touch signals generated or modified by numerous touch sensitive sensor segments.

23. The sensor assembly according to claim 20, wherein the touch sensitive sensor segment is operable to generate different electric touch signals in response to a variation of a pressure applied to the touch sensitive sensor segment.

24. The sensor assembly according to claim 20, wherein the processor is operable to assign the temporal variation of the electric touch signal to one of a plurality of predefined user gestures and wherein the processor is further operable to generate the control signal by selecting the control signal from a plurality of predefined control signals on the basis of the user gesture assigned to the temporal variation of the electric touch signal.

25. The sensor assembly according to claim 24, wherein the processor is operable to recognize at least one of:
a swipe motion of the body part across the sensing surface,
a short-term tapping motion of the body part on the sensing surface,
a multiple short-term tapping motion of the body part on the sensing surface,
a long-term tapping motion of the body part on the sensing surface and
a variable pressure applied by the body part on the sensing surface.

26. The sensor assembly according to claim 20, wherein the processor is operable to process electric touch signals of a number of touch sensitive sensor segments to ascertain a sensing area of the sensing surface being touched by the body part.

27. The sensor assembly according to claim 20, wherein the sensor element includes a planar sensing surface configured for fastening to an end face of a trigger of the injection device.

28. The sensor assembly according to claim 20, wherein the sensor element includes a tubular shaped sensing surface configured for fastening to a tubular member of the injection device.

29. The sensor assembly according to claim 20, wherein the sensor element includes a flexible sheet configured for wrapping around the tubular member of the injection device.

30. The sensor assembly according to claim 20, wherein generation of the control signal is triggered by the detection of a variation of the electric touch signals over time.

31. The sensor assembly according to claim 20, wherein submission of the control signal to the external electronic device is automatically triggered upon generating the control signal.

32. The sensor assembly according to claim 20, wherein the processor is operable to detect, to recognize, to characterize and/or to measure an operation of the injection device on the basis of the temporal variation of the electric touch signal and wherein the processor is operable to generate the control signal by selecting one of a number of predefined control signals being indicative of the detected, recognized, characterized and/or measured operation of the injection device.

33. The sensor assembly according to claim 20, wherein the processor is operable to distinguish between a dose setting operation, a dose dispensing operation and an end of dose holding operation of the injection device by processing numerous electric touch signals of numerous touch sensitive sensor segments over time when the sensor element is attached to one of the injection device and an add-on device attached to the injection device.

34. The sensor assembly according to claim 20, further comprising a clock and a memory connected to the processor and wherein the processor, the clock and the memory are operable to automatically store or to automatically monitor and/or to log a sequence of user-induced operations of the injection device by detecting or measuring variations of electric touch signals of the sensor element.

35. An injection device for injecting a dose of a medicament, the injection device comprising:
a body to accommodate a drive mechanism operable to withdraw or to expel the medicament from a medicament container,
at least one of a dose dial and a trigger actuatable by a user for injecting the dose and
a sensor assembly of claim 20 attached to or integrated into at least one of the body, the dose dial and the trigger.

36. An injection system comprising an external electronic device and an injection device, wherein the injection device comprises a body to accommodate a drive mechanism operable to withdraw or to expel the medicament from a medicament container, at least one of a dose dial and a trigger actuatable by a user for injecting the dose and a sensor assembly of claim 20 attached to or integrated into at least one of the body, the dose dial and the trigger, and wherein the external electronic device comprises:
a user interface to communicate with a user of the injection device,
a device processor connected to the user interface and operable to provide at least one of an indication and a user instruction to the user via the user interface,
a device transceiver configured to receive a control signal from the transmitter of the sensor assembly of the injection device.

37. A method of assisting a user to use an injection device by making use of an injection system of comprising an external electronic device and an injection device, wherein the injection device comprises a body to accommodate a drive mechanism operable to withdraw or to expel the medicament from a medicament container, at least one of a dose dial and a trigger actuatable by a user for injecting the dose and a sensor assembly of claim 20 attached to or integrated into at least one of the body, the dose dial and the trigger, and wherein the external electronic device comprises:
a user interface to communicate with a user of the injection device,
a device processor connected to the user interface and operable to provide at least one of an indication and a user instruction to the user via the user interface,
a device transceiver configured to receive a control signal from the transmitter of the sensor assembly of the injection device, the method including the steps of:
prompting the user to operate the injection device via the user interface of the external electronic device,
detecting of a variation of an electric touch signal over time caused by the user touching the sensing surface of the sensor element of the sensor assembly of the injection device,
generating a control signal on the basis of the temporal variation of the electric touch signal and
transmitting the control signal to the external electronic device via the transmitter of the sensor assembly.
